(19) United States
(12) Patent Application Publication
(10) Pub. No.: US 2024/0310343 A1
(43) Pub. Date: Sep. 19, 2024

(54) SPACE EVALUATION SYSTEM

(71) Applicants: TOYOTA JIDOSHA KABUSHIKI KAISHA, Toyota-shi, Aichi (JP); INTER-UNIVERSITY RESEARCH INSTITUTE CORPORATION RESEARCH ORGANIZATION OF INFORMATION AND SYSTEMS, Tachikawa-shi, Tokyo (JP)

(72) Inventors: Akinobu TOYODA, Toyota-shi, Aichi (JP); Masakazu ITO, Toyota-shi, Aichi (JP); Satoshi KATAHIRA, Nagakute-shi, Aichi (JP); Yuji MATSUO, Fukuoka-shi, Fukuoka (JP); Akinori IKEUCHI, Nagakute-shi, Aichi (JP); Ken KUROKAWA, Mishima-shi, Shizuoka (JP); Koichi HIGASHI, Mishima-shi, Shizuoka (JP); Hiroshi MORI, Mishima-shi, Shizuoka (JP)

(73) Assignees: TOYOTA JIDOSHA KABUSHIKI KAISHA, Toyota-shi, Aichi (JP); INTER-UNIVERSITY RESEARCH INSTITUTE CORPORATION RESEARCH ORGANIZATION OF INFORMATION AND SYSTEMS, Tachikawa-shi, Tokyo (JP)

(21) Appl. No.: 18/271,532

(22) PCT Filed: Jan. 14, 2022
(86) PCT No.: PCT/JP2022/001136

§ 371 Date: Jul. 10, 2023

(30) Foreign Application Priority Data
Jan. 15, 2021 (JP) 2021-005128

(51) Int. Cl. G01N 33/00

(52) U.S. Cl. CPC G01N 33/0027

(57) ABSTRACT

Provided is a novel space evaluation system capable of simply and quantitatively evaluating how close an unknown space is to a natural environment. The space evaluation system 1 comprises: a setting unit 12 for setting naturalness as an index of how close a space is to a natural environment; and an estimating unit 11 for estimating, from air quality data indicating a type of material including a microbe included in a sample collected from air in a target space to be evaluated and indicating an abundance of each material, the naturalness of the target space from which the sample has been collected.

### TECHNICAL FIELD

The present invention relates to a space evaluation system.

### BACKGROUND ART

As the awareness for the maintenance and improvement of people's health as well as their mental and physical functions increases, realization of spaces that improve labor productivity and provide a high stress-reducing effect is gaining attention. For example, it is well known that someone living together with a plant can expect to obtain a healing effect, and realization of a space into which biophilic design is incorporated and that lets one “feel as if one were in a natural forest” is expected. Biophilic design is the practice of designing a space based on the concept of biophilia that “humans have an instinctive desire to connect with nature”. In a space design such as a biophilic design, it is important to ascertain how close the space is to a natural environment.

Approaches for objectively evaluating a natural environment have been proposed. Patent Literature 1 discloses a method by which tree-trunk shape images captured of a forest area from above and spectral analysis results are analyzed to evaluate the forest area. Patent Literature 2 discloses an approach for evaluating naturality by ascertaining the state of material circulation from data on the amount of plant and data on microbial activity in a natural environment.

Evaluation approaches that focus on the degree of naturalness felt by humans have also been proposed. For example, Patent Literature 3 discloses a (space evaluating) approach wherein physiological response information when in a space in a forest and physiological response information when in a space in an urban area are acquired, and it is determined whether the space in the forest is suitable for forest bathing based on the difference in physiological response information between the two. Non Patent Literature 1 discloses a method for evaluating the degree of naturalness of a space based on evaluation items including the light and colors in an indoor space, fractal structures of a landscape, the presence or absence of living organisms in the space, and the like.

### CITATION LIST

### Patent Literature

Patent Literature 1: JP 2001-357380 A
Patent Literature 2: JP 2014-039493 A
Patent Literature 3: JP 2005-103309 A

### Non Patent Literature

Nikos A. Salingaros, “The Biophilic Healing Index Predicts Effects of the Built Environment On Our Wellbeing”, Journal of Biourbanism, 8(1/2019), p. 13-34

### SUMMARY OF INVENTION

### Technical Problem

However, the approach disclosed in Patent Literature 1 mainly involves analysis of image data captured from up in the air, and is therefore limited to evaluation by means of an image. The approach disclosed in Patent Literature 2 is not applicable if there is no soil in the target space. According to the approach disclosed in Patent Literature 3, in order to evaluate an unknown space, it is necessary to acquire relative changes in physiological response information between a plurality of different spaces and to then perform analysis, requiring significant effort and time for the evaluation. In addition, as the evaluation result is largely dependent on individual differences between the subjects providing the physiological response information, it is difficult to evaluate the space quantitatively. According to the approach disclosed in Non Patent Literature 1, because the respective evaluation items are mainly based on visual information and the evaluation is in three levels, the amount of extracted information is small. Further, because the evaluation approach is limited to indoor spaces, it is difficult to evaluate naturalness in comparison to a natural environment.

The present invention was made in view of the foregoing, and it is an object of the present invention to provide a novel space evaluation system capable of simply and quantitatively evaluating how close an unknown space to be evaluated is to a natural environment.

### Solution to Problem

In a space design such as a biophilic design, it is important to ascertain the “naturalness” as an index of how close the space is to a natural environment. The inventors have found that the naturalness of a space is affected by the quality of air present in the space (hereafter also referred to as “air quality”). In particular, the inventors have found that the naturalness of a space is greatly affected by microbes present in the air in the space.

In order to solve the problem, a space evaluation system according to the present invention includes a setting unit in which naturalness as an index of how close a space is to a natural environment is set; and an estimating unit for estimating, from air quality data indicating a type of material including a microbe included in a sample collected from air in a target space to be evaluated and indicating an abundance of each material, the naturalness of the target space from which the sample has been collected.

Thus, the space evaluation system, by simply collecting a sample from air in a target space that may be freely determined, and acquiring air quality data of the collected sample, can estimate naturalness from the air quality data alone. That is, the space evaluation system can estimate naturalness from the air quality data alone, without capturing an image of the target space from above, acquiring physiological response information in the target space, or performing sensory evaluation each time. In addition, the space evaluation system is applicable whether the target space is a space having no soil, such as an indoor space, or an outdoor space closer to a natural environment, and can estimate naturalness irrespective of the attributes of the target space. Accordingly, the space evaluation system can simply and quantitatively evaluate how close an unknown space is to a natural environment.

In a further preferred embodiment, in the setting unit, the naturalness may be set based on environment data indicating conditions of a plurality of specific spaces. The environment data may include data acquired in each of the plurality of specific spaces having different environments.

Thus, the space evaluation system can establish the naturalness as an index that enables objective evaluation of various spaces having different environments. Accordingly, the space evaluation system can accurately estimate naturalness by means of the estimating unit, and can therefore accurately evaluate how close an unknown space is to a natural environment.

In a further preferred embodiment, the environment data may include quantitative data acquired by a sensor in the specific spaces and qualitative data acquired in the specific spaces through sensory evaluation.

Thus, the space evaluation system can calculate and set naturalness by combining various data of different perspectives including quantitative data and qualitative data, and can therefore establish the naturalness as an index having high probability of enabling comprehensive evaluation from various viewpoints. In particular, because the environment data includes qualitative data acquired through sensory evaluation, the space evaluation system can establish the naturalness as an index approximating human sensory evaluation results. Thus, the space evaluation system can more accurately estimate naturalness by means of the estimating unit, and can therefore more accurately evaluate how close an unknown space is to a natural environment.

In a further preferred embodiment, the calculation of the naturalness may be machine-learned using, as training data, a data set in which the air quality data of a sample for learning collected from air in each of the plurality of specific spaces is associated with the naturalness corresponding to each of the plurality of specific spaces.

Thus, the space evaluation system can more simply and accurately estimate naturalness only from the air quality data of the target space that may be freely determined, and can therefore more simply and accurately evaluate how close an unknown space is to a natural environment.

In a further preferred embodiment, the air quality data may be acquired by analyzing, by means of an analysis device, a sample collected by a collecting device. In the setting unit, one or both of the air quality data of the material present in the collecting device before the sample is collected and the air quality data of the material present in the analysis device before the sample is analyzed may be set as the air quality data of a negative control sample. The estimating unit may estimate a contaminated proportion of the air quality data of the negative control sample that contaminates the air quality data of the sample collected in the target space, and may estimate the naturalness of the target space from the air quality data of the target space from which the air quality data of the negative control sample has been removed.

Thus, the space evaluation system can estimate naturalness from the true air quality data of the collected sample. Accordingly, the space evaluation system can more accurately estimate naturalness by means of the estimating unit, and can therefore more accurately evaluate how close an unknown space is to a natural environment.

### Advantageous Effects of Invention

According to the present invention, it is possible to provide a novel space evaluation system capable of simply and quantitatively evaluating how close an unknown space to be evaluated is to a natural environment.

### BRIEF DESCRIPTION OF DRAWINGS

FIG. 1 illustrates the configuration of a space evaluation system.

FIG. 2 illustrates an example of environment data.

FIG. 3 illustrates a BPS calculation approach.

FIG. 4 illustrates the result of verifying the validity of the BPS calculation approach.

FIG. 5 illustrates a procedure for acquiring microbial community structure data.

FIG. 6 illustrates a graphical model representing a BPS estimation model.

FIG. 7 illustrates topics and n parameters extracted by machine learning related to the BPS estimation model.

FIG. 8 illustrates contaminated proportions of microbial community structure data of NC samples that contaminates the microbial community structure data of each sample.

FIG. 9 illustrates mixing proportions of topics in each of the samples illustrated in FIG. 8.

FIG. 10 illustrates the result of the validation of the BPS estimation model.

FIG. 11 illustrates the result of estimating the BPS of target spaces using the BPS estimation model.

FIG. 12 illustrates a graphical model representing an NC estimation model by LDAnc.

FIG. 13 illustrates an example of the result of verifying the estimation accuracy of the NC estimation model by LDAnc.

FIG. 14 illustrates another example of verifying the estimation accuracy of the NC estimation model by LDAnc.

### DESCRIPTION OF EMBODIMENTS

In the following, embodiments of the present invention will be described with reference to the drawings. Configurations referred to by like reference signs in the respective embodiments have like or similar functions in the respective embodiments unless otherwise noted, and their description may be omitted.

### [Configuration of Space Evaluation System]

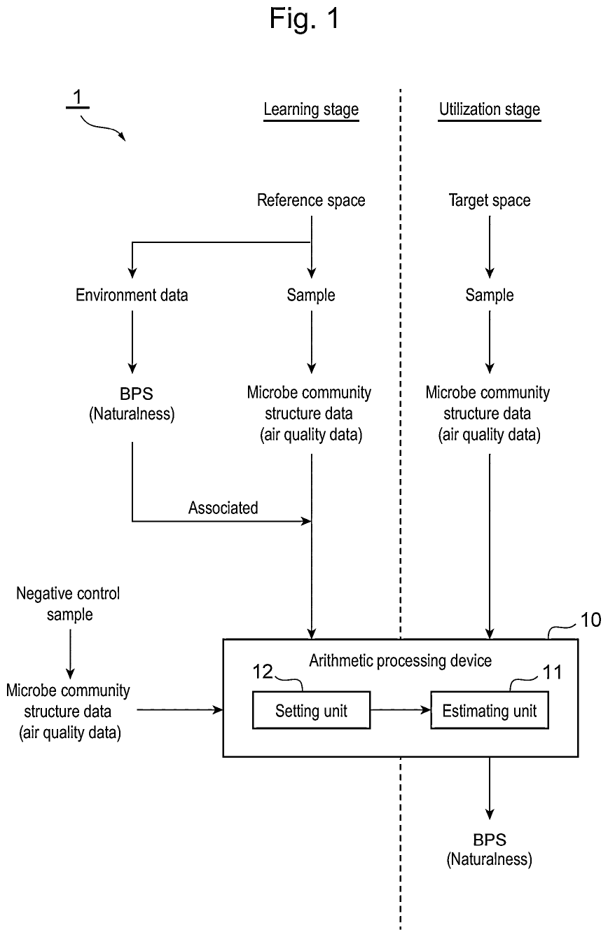

With reference to FIG. 1, the configuration of a space evaluation system 1 will be described. FIG. 1 illustrates the configuration of the space evaluation system 1.

The space evaluation system 1 is a system for evaluating how close various spaces, including an outdoor space, such as a forest or an urban area, and an indoor space such as an office or a residence, are to a natural environment. The space evaluation system 1 is effective in realizing a space incorporating the biophilic design. In a space design for constructing a space for coexistence with plants that allows one to experience nature, such as a biophilic design, it is important to ascertain “naturalness” as an index of how close the space is to a natural environment. Further, in addition to sensory stimulations such as visual perception and auditory perception, people are also affected by the air quality of a space. In such space designs, it is important to evaluate the naturalness of a space by also focusing on air quality.

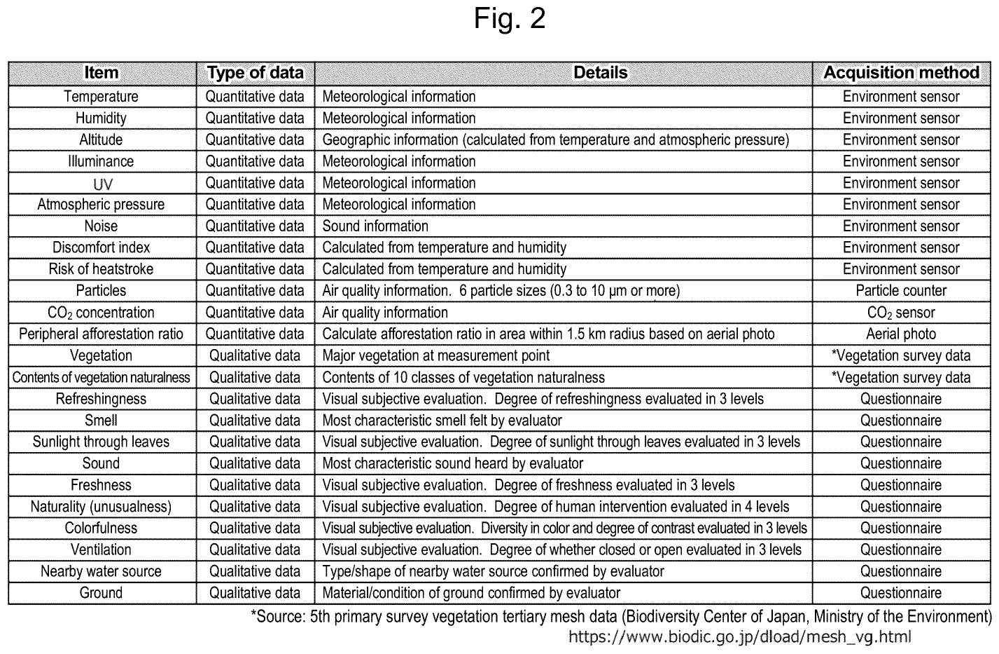

The present embodiment introduces a biophilic score (hereafter also referred to as “BPS”) as the naturalness of a space focusing also on air quality. The BPS is calculated by analyzing “environment data” indicating the condition of a space, such as its temperature and humidity, using a statistical approach. Details of the environment data and the calculation of the BPS will be described below with reference to FIG. 2 to FIG. 4.

The space evaluation system 1 estimates the BPS of an unknown space to be evaluated (hereafter also referred to as a “target space”) from data indicating the air quality (hereafter also referred to as “air quality data”) of the target space. The target space is a space that may be freely determined, whether an indoor space or an outdoor space. The air quality data of the target space is data that indicates the type of materials including microbes contained in a sample collected from the air in the target space, and that indicates the abundance of each of the materials (relative abundance).

In addition to microbes, examples of the materials included in the samples used in the space evaluation system 1 include inorganic gases, volatile organic compounds, and allergen. Microbes are present in various environments, and are known to affect material circulation and the health state and the like of a host, for example. Microbes present in the air in the target space affect the quality of the air in the target space. In the present embodiment, attention is focused on microbes as the materials included in the samples used in the space evaluation system 1, and microbial community structure data of the target space is adopted as the air quality data of the target space. The microbial community structure data of the target space is data that indicates the type of microbes (microbial strains) belonging to the microbial community included in a sample collected from the air in the target space, and the abundance (relative abundance) of each of the microbes.

As illustrated in FIG. 1, the space evaluation system 1 is provided with an arithmetic processing device 10. The arithmetic processing device 10 is comprised of hardware including a processor and a storage device, and software including a program. In the arithmetic processing device 10, the processor performs a program stored in the storage device to implement various functions of the space evaluation system 1. Although not illustrated, the space evaluation system 1 may be provided with an input device for inputting data and the like into the arithmetic processing device 10, and an output device for outputting an arithmetic processing result from the arithmetic processing device 10. Further, the space evaluation system 1 may be provided with a communication device for performing communications with an external apparatus.

The arithmetic processing device 10 includes an estimating unit 11 for estimating the BPS of a target space from the microbial community structure data of the target space, and a setting unit 12 in which the microbial community structure data and the BPS of reference spaces are set. The estimating unit 11 is comprised of a mathematical model (hereafter also referred to as an “estimation model”) for estimating the BPS of a target space from the microbial community structure data of the target space.

In the present embodiment, the estimating unit 11 has machine-learned to calculate the BPS with respect to the microbial community structure data of the target space, using, as training data, a data set in which the microbial community structure data of samples for learning collected from the air in each of a plurality of reference spaces is associated with a BPS corresponding to each of the plurality of reference spaces. Each of the plurality of reference spaces is a predetermined space for collecting the samples for learning. In the present embodiment, the spaces adopted as the plurality of reference spaces include various outdoor spaces such as a forest, a park, and an urban area; various indoor spaces such as an office, a laboratory, and a residence; and an experimentally fabricated indoor afforestation space. The reference spaces are an example of a “specific space” set forth in the claims.

Because the estimating unit 11 has machine-learned to calculate the BPS with respect to the microbial community structure data of the target space using the data set as training data, the space evaluation system 1 can more simply and accurately estimate the BPS only from the air quality data of the target space. Thus, the space evaluation system 1 can more simply and accurately evaluate how close an unknown space is to a natural environment.

A procedure for constructing the BPS estimation model constituting the estimating unit 11 will be described. In the estimation model learning stage, first, a sample for learning is collected from the air in each of a plurality of predetermined reference spaces. The structure of a microbial community included in each of the collected samples is analyzed to acquire the microbial community structure data for each of the plurality of reference spaces. Also, environment data is acquired in each of the plurality of reference spaces. Based on the acquired environment data, a BPS is calculated. Then, the microbial community structure data for each of the plurality of reference spaces is associated with the BPS corresponding to each of the plurality of reference spaces to create a data set. The created data set is set in the setting unit 12. The setting unit 12 sets the data set in the estimation model as training data, and trains the estimation model by machine learning to calculate the BPS with respect to the microbial community structure data of the target space. In this way, a trained estimation model is constructed. In the space evaluation system 1, the processing for implementing the setting of the training data and machine learning with respect to the estimation model may be performed by the setting unit 12.

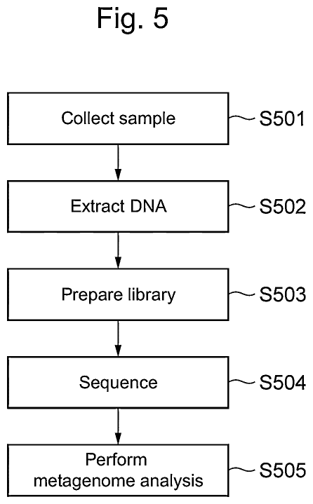

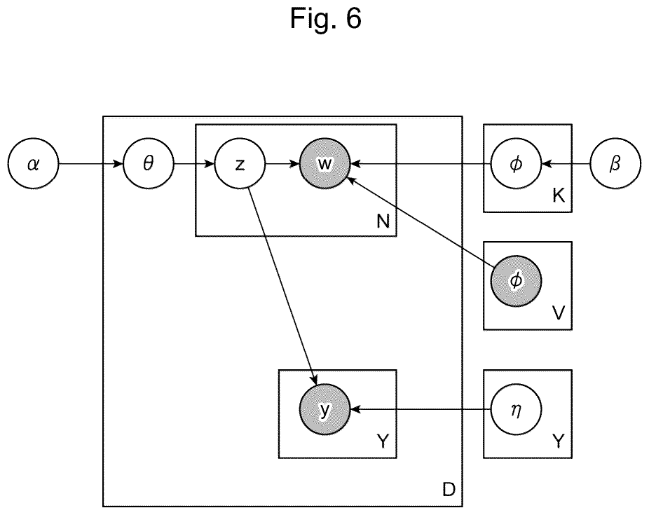

In the estimation model learning stage, in addition to the data set, microbial community structure data of a negative control sample (hereafter also referred to as “NC sample”) is set in the estimation model. The NC sample essentially is a material that exists in the air of neither the reference spaces nor the target space. The NC sample is a material that could enter during the process of acquiring the microbial community structure data by collecting samples from the air in the reference spaces or the target space. The NC sample is, for example, a material present in a collecting device, such as an air sampler used for collecting a sample from the air; in an analysis device for the collected sample; or in a reagent and the like. In the present embodiment, microbial community structure data of microbes present in the collecting device before a sample is collected, and/or microbial community structure data of microbes present in the analysis device before a sample is analyzed is set in the setting unit 12 in advance as the microbial community structure data of the NC sample. The setting unit 12 sets the microbial community structure data of the NC sample in the estimation model, and then performs the machine learning using the data set and the microbial community structure data of the NC sample to construct the trained estimation model. The acquisition of the microbial community structure data will be described below with reference to FIG. 5. The details of the machine learning concerning the estimation model will be described below with reference to FIG. 6 to FIG. 11.

A procedure for estimating the BPS of the target space by the BPS estimation model constituting the estimating unit 11 will be described. In the BPS estimation model utilization stage, first, a sample is collected from the air in the target space. The structure of microbial communities included in the collected sample is analyzed to acquire the microbial community structure data of the target space. The microbial community structure data of the target space is then input into the trained BPS estimation model to estimate the BPS of the target space. Specifically, in the trained BPS estimation model, the contaminated proportion of the microbial community structure data of the NC sample that contaminates the microbial community structure data of the sample collected in the target space is estimated, and the BPS of the target space is estimated from the microbial community structure data of the target space from which the microbial community structure data of the NC sample has been excluded.

Accordingly, the space evaluation system 1 can estimate the BPS from the true microbial community structure data of the sample collected in the target space. Conventionally, it has been difficult to appropriately estimate the contaminated proportion of the microbial community structure data of the NC sample, and therefore it has been difficult to acquire the true microbial community structure data of the sample collected in the target space. The space evaluation system 1 can estimate the contaminated proportion of the microbial community structure data of the NC sample that contaminates the microbial community structure data of the target space, and can estimate the BPS from the true microbial community structure data of the collected sample. Thus, the space evaluation system 1 can more accurately estimate the BPS by means of the estimating unit 11, and can therefore more accurately evaluate how close an unknown space is to a natural environment.

It is noted that the estimating unit 11 is not limited to an estimation model constructed by machine learning as described above. The estimating unit 11 may be comprised of a relational expression, a table, a graph, or the like describing the relationship between the microbial community structure data acquired in each of a plurality of reference spaces and the BPS.

### [Calculation of BPS]

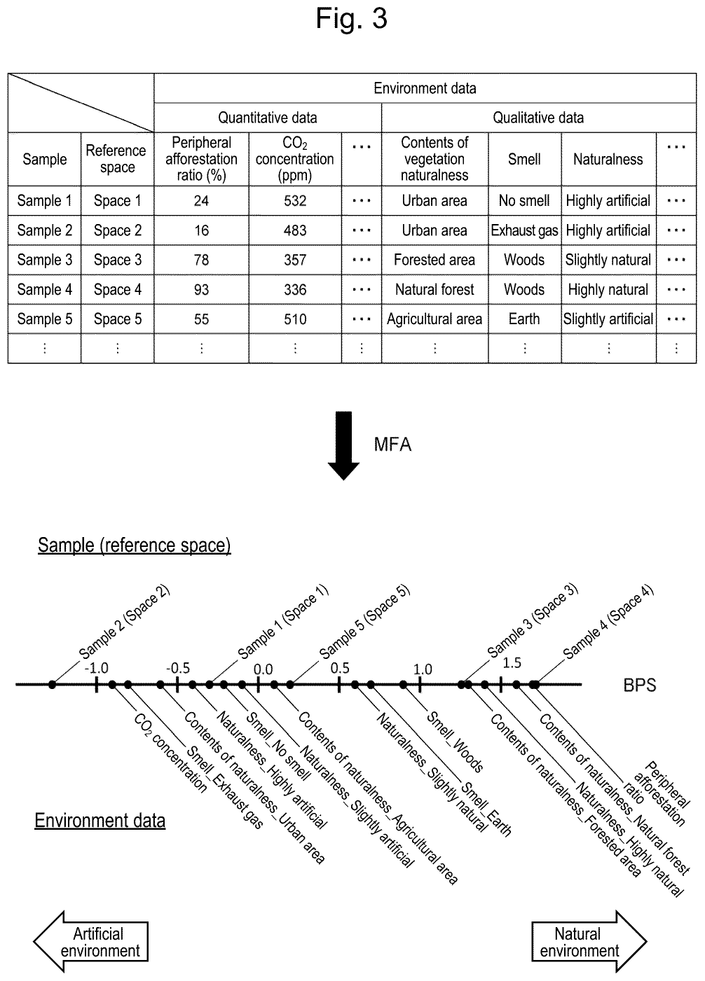

With reference to FIG. 2 to FIG. 4, the BPS calculation approach will be described. FIG. 2 illustrates an example of environment data. FIG. 3 is a diagram illustrating the BPS calculation approach.

The BPS is calculated based on environment data acquired in each of a plurality of reference spaces. The environment data is data acquired in each of a plurality of reference spaces having different environments. The plurality of reference spaces having different environments may comprise, for example, a plurality of reference spaces having different numbers of artificial objects, such as concrete buildings, or natural objects, such as forests. In the setting unit 12, the BPS calculated based on environment data indicating the condition of each of the plurality of reference spaces are stored.

Thus, the space evaluation system 1 can establish the BPS as an index that enables objective evaluation of a plurality of reference spaces having different environments. Accordingly, the space evaluation system 1 can accurately estimate naturalness by means of the estimating unit 11, and can therefore accurately evaluate how close an unknown space is to a natural environment.

One environment data acquired in one reference space includes, as illustrated in FIG. 2, a plurality of quantitative data items acquired by various sensors in the reference space, and a plurality of qualitative data items acquired through sensory evaluation, such as a questionnaire survey, in the reference space.

Thus, the space evaluation system 1 can calculate and set the BPS by combining various data of different perspectives including quantitative data and qualitative data. Accordingly, the space evaluation system 1 can establish the BPS as an index having high probability of enabling comprehensive evaluation from various viewpoints. In particular, because the environment data includes qualitative data acquired through sensory evaluation, the space evaluation system 1 can establish the naturalness as an index approximating human sensory evaluation results. Accordingly, the space evaluation system 1 can more accurately estimate naturalness by means of the estimating unit 11, and can therefore more accurately evaluate how close an unknown space is to a natural environment.

The acquired environment data is associated with the sample collected in the reference space in which the environment data has been acquired, and is stored in a table shown at the top of FIG. 3. In this table, as shown at the top of FIG. 3, the quantitative data and the qualitative data are stored separately.

The BPS is calculated by performing multiple factor analysis (MFA) on the environment data. Specifically, first, principal component analysis is performed with respect to the quantitative data included in the environment data, and also multiple correspondence analysis is performed with respect to the qualitative data included in the environment data. Then, singular value decomposition is performed with respect to each. As a scaling process for unifying the scales between the data, the whole of the quantitative data is divided by a first singular value obtained by the singular value decomposition of the quantitative data, and also the whole of the qualitative data is divided by a first singular value obtained by the singular value decomposition of the qualitative data. A table in which the quantitative data on which the scaling process has been performed is stored, and a table in which the qualitative data on which the scaling process has been performed is stored are integrated. Principal component analysis is performed with respect to the entire data stored in the integrated table. In this way, multi-dimensional environment data including a plurality of quantitative data items and a plurality of qualitative data items is dimensionally compressed as one-dimensional continuous-value data illustrated by the number line shown at the bottom of FIG. 3.

On the upper side of the number line illustrated in FIG. 3, the samples collected in the respective reference spaces are plotted. On the lower side of the number line shown in FIG. 3, a plurality of quantitative data items and a plurality of qualitative data items included in the environment data acquired in the respective reference spaces are plotted in a mixed manner. On the number line illustrated in FIG. 3, more “artificial” environment data appears toward the negative direction (toward left), and more “natural” environment data appears toward the positive direction. The number line illustrated in FIG. 3 indicates an index relatively expressing whether the space is closer to an artificial environment or closer to a natural environment. In the present embodiment, the one-dimensional continuous-value data indicated by the number line of FIG. 3 is defined as the BPS. Thus, the BPS is calculated based on the environment data acquired in each of a plurality of reference spaces. The space evaluation system 1 may include a calculation unit for calculating the BPS.

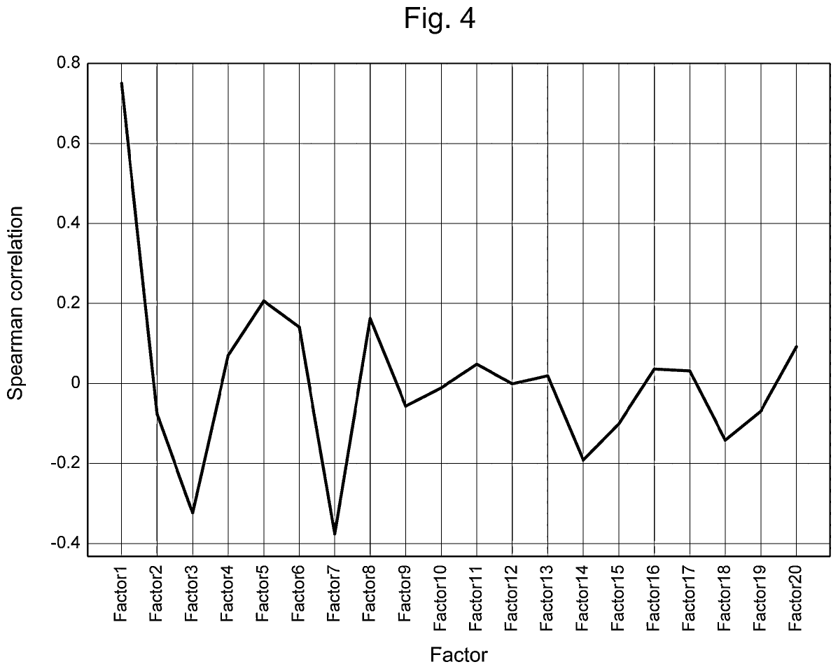

FIG. 4 illustrates the result of verifying the validity of the BPS calculation approach.

The graph shown in FIG. 4 indicates the result of computation of the Spearman correlation between Factor 1 to Factor 20 obtained by performing multiple factor analysis with respect to the environment data, and the results of a vegetation naturalness survey published by the Environment Ministry's Nature Conservation Bureau. As shown in FIG. 4, the Spearman correlation value for Factor 1 shows a high value of about 0.75. The Spearman correlation values for Factor 2 to Factor 20 show lower values with significant differences from the Spearman correlation value for Factor 1. Thus, it is considered valid to define, as the BPS, data obtained by dimensional compression of multi-dimensional environment data into Factor 1 through multiple factor analysis.

It is noted that, while the environment data shown in FIG. 2 includes “peripheral afforestation ratio” as one of the quantitative data, normalized difference vegetation index (NDVI) may be adopted instead of the “peripheral afforestation ratio”. The NDVI is a vegetation index calculated by acquiring the reflectances of plants with respect to the electromagnetic waves of the visible and near-infrared regions from an artificial satellite or the like. In this way, accurate afforestation ratios around the reference spaces can be calculated.

### [Acquisition of Microbial Community Structure Data]

With reference to FIG. 5, acquisition of microbial community structure data will be described. FIG. 5 illustrates a procedure for acquiring microbial community structure data.

In step S501, first, a sample is collected from the air in a reference space. Specifically, a collecting device, such as the MD8 Airscan or AirPort from Sartorius AG and a gelatin filter are used to suction 3000 L of the air, and a microbial community in the air is caused to be adsorbed onto the gelatin filter.

In step S502, DNA is extracted from the collected sample. Specifically, the gelatin filter is dissolved and filtered, and DNA is extracted using DNeasy PowerWater Kit from QIAGEN.

In step S503, a library is prepared. Specifically, a primer targeting the V1-V2 region of 16S rRNA is used, and PCR amplification is performed in accordance with the standard protocol of Illumina, Inc. to prepare the library.

In step S504, DNA sequencing is performed. Specifically, the iSeq 100 sequencer from Illumina, Inc. is used, and 2×150 bp paired-end sequencing is performed.

In step S505, metagenome analysis is performed. Taxonomic composition data of microbial communities is obtained by shotgun metagenomic sequencing or 16S rRNA amplicon sequencing. Especially in the case of 16S rRNA amplicon sequencing, forward reads after adapter sequence removal are analyzed by Qiime2. In this way, the microbial community structure data of the sample collected from the air in the reference space is acquired.

It is noted that a procedure for acquiring the microbial community structure data of a sample collected from the air in the target space also involves steps similar to the step S501 to step S505 described above. Further, a procedure for acquiring the microbial community structure data of an NC sample also involves steps similar to the step S502 to step S505 described above, with the exception that in step S501, the sample is collected from the air in the reference space or the target space.

### [Machine Learning Related to BPS Estimation Model]

With reference to FIG. 6 to FIG. 11, machine learning related to the BPS estimation model will be described. FIG. 6 illustrates a graphical model representing the BPS estimation model.

As an approach for learning conversion from multivariate data, such as microbial community structure data, into numerical value data, such as the BPS, a number of machine learning approaches are available. Among others, non-linear transform approaches such as the random forest and deep learning are known to have high prediction accuracy, and there are many utilization examples. However, these non-linear transform approaches are generally difficult in terms of interpretation of the transform rules. Also, in the present embodiment, it is preferable to be able to construct an estimation model in which the relationship between the microbial community structure data and the BPS is clearly indicated. For example, it is preferable to be able to construct an estimation model that clearly indicates what partial community (constituent unit of a microbial community; hereafter referred to as “sub-community”) should be added to or removed from the microbial community structure data to change the BPS. Further, the process of acquiring the microbial community structure data is essentially a probabilistic phenomenon. Generally, it is impossible to directly observe a “true microbial community” included in a sample, and the microbial community structure data is always acquired by probabilistic sampling from the sample. With a deterministic approach such as deep learning, it is not easy to capture such probabilistic property of data.

Accordingly, in the present embodiment, as a machine learning approach related to the BPS estimation model, supervised Latent Dirichlet Allocation (hereafter also referred to as “sLDA”) is adopted, which is one of topic models. Also, in the present embodiment, the microbial community structure data of the NC samples is set in the estimation model in advance. The sLDA is a modeling approach for simultaneously learning auxiliary information and count data to extract “topics”. In the sLDA, each of the topics is linked with a “regression coefficient of auxiliary information” (one-dimensional continuous value). It is noted that while in the present embodiment sLDA is adopted as the machine learning approach related to the BPS estimation model, other approaches may be adopted.

The variables used in mathematical expressions describing the BPS estimation model are defined as follows:
K: Number of unknown topics.
V: Number of NC samples.
ϕ_{k=1 . . . K}: Community structure of unknown topic (relative abundance of microbial strain).
ϕ_{k=K+1 . . . K+V}: Community structure of NC sample.
Y: Number of dimensions of response variable. Since the BPS is one-dimensional numerical value data, Y=1.
y_{d=1 . . . D}: Response variable. Some numerical value information given to the sample. In the present embodiment, the BPS.
η_{k=1 . . . K}: K-dimension column vector of a “weight” parameter for each topic for converting a topic composition into a response variable. Regression coefficient.
D: Number of samples.
T: Number of microbial strains that can be annotated.
N: Number of DNA sequences included in sample d. When emphasizing sample d in particular, noted N_{d}.
Nₖ: Number of DNA sequences assigned to topic k.
N_{dk}: Number of DNA sequences assigned to topic k included in sample d.
Nₜₖ: Number of DNA sequences assigned to topic k and microbial strain t.
w_{dn}: Annotation of microbial strain given to DNA sequence n of sample d.
z_{dn}: Latent topic of DNA sequence n of sample d.
θ_{d=1 . . . D}: Topic composition of sample d (mixing proportions (relative abundance) of topic and NC community).
α: Parameter (prior weight) of Dirichlet distribution of topic composition. Vector of length K+V.
β: Parameter (prior weight) of Dirichlet distribution of community structure. Vector of length T.

The generative process of the BPS estimation model is as follows:
1. θ_{d}˜Dir(α): The topic composition θ_{d} of sample d is sampled from Dirichlet distribution Dir(a) having α as a parameter.
2. ϕₖ˜Dir(β): The community structure ϕₖ of topic k is sampled from Dirichlet distribution Dir(β) having β as a parameter.
3. ηₖ˜ [symbol] (0.0,10.0): The initial value of weight for each topic is sampled from a Gaussian distribution. It is noted that the average parameter and distributed parameter of the Gaussian distribution are fixed to (0,10).
4. Concerning DNA sequence n of sample d
4-1. z_{dn}˜Categorical(θ_{d}): A topic number or a NC sample number of the DNA sequence are sampled from a categorical distribution.
4-2. w_{dn}˜Categorical(ϕₖ): A microbial strain is sampled from a corresponding topic or NC sample.
5. Concerning sample d

$$\overline{{z}_{d}} = ( 1 / N ) {\sum }_{n = 1}^{N} {z}_{d n}$$

5-2. y˜ [symbol] (η^{T}z̅_̅{̅d̅}̅, 1.0): A response variable is sampled and generated from a Gaussian distribution having, as a mean, a value obtained by multiplying a latent topic composition by η parameter. It is noted that the distributed parameter of the Gaussian distribution is fixed to 1.0.

Bayesian inference of an unknown parameter is performed. First, a conventional sLDA is considered in which the microbial community structure data of an NC sample is not set in the estimation model. The joint probability of the estimation model is described as follows:

$$p ( θ , ϕ , z , w , η , y \vert α , β ) = {\prod }_{d = 1}^{D} p ( {θ}_{d} \vert α ) {\prod }_{k = 1}^{K} p ( {ϕ}_{k} \vert β ) {\prod }_{n = 1}^{{N}_{d}} p ( {z}_{dn} \vert {θ}_{d} ) p ( {w}_{dn} \vert {ϕ}_{{z}_{dn}} ) \times {\prod }_{k = 1}^{K} p ( {η}_{k} \vert 0 , 10 ) {\prod }_{d = 1}^{D} p ( {y}_{d} \vert η , z ) = {\prod }_{d = 1}^{D} \frac{{\prod }_{k = 1}^{K} {θ}_{dk}^{α - 1}}{B ( α )} {\prod }_{k = 1}^{K} {θ}_{dk}^{{N}_{dk}} {\prod }_{k = 1}^{K} \frac{{\prod }_{t = 1}^{T} {ϕ}_{tk}^{β - 1}}{B ( β )} {\prod }_{t = 1}^{T} {ϕ}_{tk}^{{N}_{tk}} \times {\prod }_{k = 1}^{K} p ( {η}_{k} \vert 0 , 10 ) {\prod }_{d = 1}^{D} p ( {y}_{d} \vert η , z ) = ( {\prod }_{d = 1}^{D} \frac{{\prod }_{k = 1}^{K} {( {θ}_{dk} )}^{{N}_{dk} + α - 1}}{B ( α )} ) ( {\prod }_{k = 1}^{K} \frac{{\prod }_{t = 1}^{T} {( {ϕ}_{tk} )}^{{N}_{tk} \_β - 1}}{B ( β )} ) \times {\prod }_{k = 1}^{K} p ( {η}_{k} \vert 0 , 10 ) {\prod }_{d = 1}^{D} p ( {y}_{d} \vert η , z ) = ( {\prod }_{d = 1}^{D} \frac{B ( {N}_{d} + α )}{B ( α )} Dir ( {N}_{d} + α ) ) ( {\prod }_{k = 1}^{K} \frac{B ( {N}_{k} + β )}{B ( β )} Dir ( {N}_{k} + β ) ) \times {\prod }_{k = 1}^{K} p ( {η}_{k} \vert 0 , 10 ) {\prod }_{d = 1}^{D} p ( {y}_{d} \vert η , z )$$

where B is a multinomial beta function. Integrating out with respect to θ,ϕ results in the following description:

$$p ( z , w , η , y \vert α , β ) = {\prod }_{d = 1}^{D} \frac{B ( {N}_{d} + α )}{B ( α )} {\prod }_{k = 1}^{K} \frac{B ( {N}_{k} + β )}{B ( β )} \times {\prod }_{k = 1}^{K} p ( {η}_{k} \vert 0 , 10 ) {\prod }_{d = 1}^{D} p ( {γ}_{d} \vert η , z )$$

N_{d} is the number of DNA sequences in the sample d, and Nₖ is the number of DNA sequences assigned to the topic k. What is desired to be determined is the posterior probability for z, η, as described below:

$$p ( z , η \vert w , y , α , β ) = \frac{p ( z , η , w , y \vert α , β )}{{\sum }_{{z}^{′}} \int p ( {z}^{′} , {η}^{′} {w}^{′} , y \vert α , β ) d {η}^{′}}$$

Since the computation of the denominator is intractable, the posterior distribution is approximated by Gibbs sampling.

The full conditional distribution at the topic z_{dn} of the DNA sequence n of the sample d is described as follows:

$$p ( {z}_{dn} = k \vert {z}_{\backslash dn} , w , η , y , α , β ) ∝ p ( {z}_{dn} = k , {z}_{\backslash dn} , w , η , y \vert α , β ) ∝ {\prod }_{k = 1}^{K} B ( {N}_{k} + β ) {\prod }_{d = 1}^{D} B ( {N}_{a} + α ) \times {\prod }_{d = 1}^{D} 𝒩 ( {y}_{d} \vert {η}^{T} {z}_{d} , 1. )$$

Initially, the term Πₖ₌₁^{K}B(Nₖ+β) is described as follows:

$${\prod }_{k = 1}^{K} N ( {N}_{k} + β ) = ( {\prod }_{{k}^{′} = 1 , {k}^{′} \neq k}^{K} B ( {N}_{{k}^{′}} + β ) ) B ( {N}_{k} + β ) = ( {\prod }_{{k}^{′} = 1 , {k}^{′} \neq k}^{K} B ( {N}_{{k}^{′}} + β ) ) \frac{{\prod }_{t = 1}^{T} Γ ( {N}_{tk} + β )}{Γ ( {\sum }_{t = 1}^{T} ( {N}_{tk} + β ) )} = ( {\prod }_{{k}^{′} = 1 , {k}^{′} \neq k}^{K} B ( {N}_{{k}^{′}} + β ) ) \frac{{\prod }_{t = 1 , t \neq {w}_{dn}}^{T} Γ ( {N}_{tk} + β )}{Γ ( {\sum }_{t = 1}^{T} ( {N}_{tk} + β ) )} Γ ( {N}_{( {w}_{dn} ) k} + β ) = ( {\prod }_{{k}^{′} = 1 , {k}^{′} \neq k}^{K} B ( {N}_{{k}^{′} \backslash {z}_{dn}} + β ) ) \frac{{\prod }_{t = 1 , t \neq {w}_{dn}}^{T} Γ ( {N}_{tk \backslash {z}_{dn}} + β )}{Γ ( {\sum }_{t = 1}^{T} ( {N}_{tk \backslash {z}_{dn}} + β ) + 1} Γ ( {N}_{( {w}_{dn} ) k \backslash {z}_{dn}} + β + 1 ) = ( {\prod }_{{k}^{′} = 1 , {k}^{′} \neq k}^{K} B ( {N}_{{k}^{′} \backslash {z}_{dn}} + β ) ) \frac{{\prod }_{t = 1 , t \neq {w}_{dn}}^{T} Γ ( {N}_{tk \backslash {z}_{dn}} + β )}{Γ ( {\sum }_{t = 1}^{T} ( {N}_{tk \backslash {z}_{dn}} + β ) ) {\sum }_{t = 1}^{T} ( {N}_{tk \backslash {z}_{dn}} + β )} \times Γ ( {N}_{( {w}_{dn} ) k \backslash {z}_{dn}} + β ) ( {N}_{( {w}_{dn} ) k \backslash {z}_{dn}} + β ) = ( {\prod }_{{k}^{′} = 1 , {k}^{′} \neq k}^{K} B ( {N}_{{k}^{′} \backslash {z}_{dn}} + β ) ) \frac{{\prod }_{t = 1}^{T} Γ ( {N}_{tk \backslash {z}_{dn}} + β )}{Γ ( {\sum }_{t = 1}^{T} ( {N}_{tk \backslash {z}_{dn}} + β ) )} \frac{( {N}_{( {w}_{dn} ) k \backslash {z}_{dn}} + β}{{\sum }_{t = 1}^{T} ( {N}_{tk \backslash {z}_{dn}} + β )} = ( {\prod }_{{k}^{′} = 1 , {k}^{′} \neq k}^{K} B ( {N}_{{k}^{′} \backslash {z}_{dn}} + β ) ) B ( {N}_{k \backslash {z}_{dn}} + β ) \frac{( {N}_{( {w}_{dn} ) k \backslash {z}_{dn}} + β}{{\sum }_{t = 1}^{T} ( {N}_{tk \backslash {z}_{dn}} + β )} = ( {\prod }_{{k}^{′} = 1 , {k}^{′} \neq k}^{K} B ( {N}_{{k}^{′} \backslash {z}_{dn}} + β ) ) \frac{( {N}_{( {w}_{dn} ) k \backslash {z}_{dn}} + β )}{{\sum }_{t = 1}^{T} ( {N}_{tk \backslash {z}_{dn}} + β )} ∝ \frac{( {N}_{( {w}_{dn} ) k \backslash {z}_{dn}} + β )}{{\sum }_{t = 1}^{T} ( {N}_{tk \backslash {z}_{dn}} + β )} = \frac{{N}_{( {w}_{dn} ) k \backslash {z}_{dn}} + β}{{N}_{k \backslash {z}_{dn}} + T β}$$

where the \z_{dn} added to the variables means a count obtained by removing z_{dn} with respect to the corresponding numerical values. Also, the property Γ(x+1)=xΓ(x) of a gamma function is utilized.

Likewise, the term Π_{d=1}^{D}B(N_{d}+α) is also described as follows:

$${\prod }_{d = 1}^{D} B ( {N}_{d} + α ) ∝ \frac{( {N}_{dk \backslash {z}_{dn}} + α )}{{\sum }_{k = 1}^{K} ( {N}_{dk \backslash {z}_{dn}} + α )} = \frac{{N}_{dk \backslash {z}_{dn}} + α}{{N}_{d \backslash {z}_{dn}} + k α}$$

Finally, the term Π_{d=1}^{D} [symbol] (y_{d}|η^{T}z_{d}, 1.0) is also computed as follows:

$${\prod }_{d = 1}^{D} 𝒩 ( {y}_{d} \vert {η}^{T} \overline{{z}_{d}} , 1. ) = {\prod }_{{d}^{′} = 1}^{D} exp ( - \frac{{( {y}_{{d}^{′}} - {η}^{T} \overline{{z}_{{d}^{′}}} )}^{2}}{2} ) ∝ {\prod }_{{d}^{′} = 1}^{D} exp ( \frac{2 {y}_{{d}^{′}} {η}^{T} \overline{{z}_{{d}^{′}}} - {( {η}^{T} \overline{{z}_{{d}^{′}}} )}^{2}}{2} ) = {\prod }_{{d}^{′} = 1}^{D} exp ( \frac{2 {y}_{{d}^{′}} ( {η}^{T} \overline{{z}_{{d}^{′} \backslash {z}_{dn}}} + {δ}_{d , {d}^{′}} \frac{{η}_{k}}{{N}_{d}} - {( {η}^{T} \overline{{z}_{{d}^{′} \backslash {z}_{dn}}} + {δ}_{d , {d}^{′}} \frac{{η}_{k}}{{N}_{d}} )}^{2}}{2} ) = {\prod }_{{d}^{′} = 1}^{D} exp ( \frac{2 {y}_{{d}^{′}} {η}^{T} \overline{{z}_{{d}^{′} \backslash {z}_{dn}}} - {( {η}^{T} \overline{{z}_{{d}^{′} \backslash {z}_{dn}}} )}^{2}}{2} ) exp ( \frac{1}{2} \frac{{η}_{k}}{{N}_{d}} ( 2 [ {y}_{d} - {η}^{T} \overline{{z}_{d \backslash {z}_{dn}}} ] - \frac{{η}_{k}}{{N}_{d}} ) ) ∝ exp ( \frac{1}{2} \frac{{η}_{k}}{{N}_{d}} ( 2 [ {y}_{d} - {η}^{T} \overline{{z}_{d \backslash {z}_{dn}}} ] - \frac{{η}_{k}}{{N}_{d}} ) )$$

where δ_{d,d′} is the Kronecker delta.

From the above, the full conditional distribution of the topic z_{dn} of the DNA sequence n of the sample d is described as follows:

$$p ( {z}_{dn} = k \vert {z}_{\backslash dn} , w , η , y , α , β ) ∝ ∝ \frac{{N}_{( {w}_{dn} ) k \backslash {z}_{dn}} + β}{{N}_{k \backslash {z}_{dn}} + T β} \times \frac{{N}_{dk \backslash {z}_{dn}} + α}{{N}_{d \backslash {z}_{dn}} + K α} \times exp ( \frac{1}{2} \frac{{η}_{k}}{{N}_{d}} ( 2 [ {y}_{d} - {η}^{T} \overline{{z}_{d \backslash {z}_{dn}}} ] - \frac{{η}_{k}}{{N}_{d}} ) ) \qquad ( 1 )$$

Next, the full conditional distribution of the weight parameter ηₖ of the topic k is considered. While a conditional distribution can be strictly determined for η, it is possible to show that, as a simple approximation of a Bayesian linear regression model, the distribution is centered around the least squares solution of the following equation:

$$γ = {Z}^{T} η \qquad ( 2 )$$

where Z is the matrix Z=(z₁ . . . . z_{D}) such that each column is the composition z̅_̅{̅d̅}̅(d∈{1 . . . . D}) of a topic assignment at that point in time of the sample d.

By the derivation up to this point, a method for updating z_{dn},η in each step of Gibbs sampling has been obtained. In implementation, first, random topics are assigned to all DNA sequences of all samples, and all z_{dn} are sampled and updated according to equation (1), and η is updated by solving equation (2). This is repeated until the joint probability of the entire model converges.

In the present embodiment, in addition to the learning of sLDA, the microbial community structure data of the NC samples is set in the estimation model in advance. In this case, what needs to be modified in the update equation of Gibbs sampling is the first term on the right-hand side of equation (1). When the microbial community structure data of the NC samples is set in the estimation model in advance, since the microbial community structure data of the NC samples is fixed at all times during the process of learning, equation (1) is modified as follows:

$$p ( {z}_{dn} = k \vert {z}_{\backslash dn} , w , η , y , α , β ) ∝ {L}_{k} \times \frac{{N}_{dk \backslash {z}_{dn}} + α}{{N}_{d \backslash {z}_{dn}}} \times exp ( \frac{1}{2} \frac{{η}_{k}}{{N}_{d}} ( 2 [ {y}_{d} - {η}^{T} \overline{{z}_{d \backslash {z}_{dn}}} ] - \frac{{η}_{k}}{{N}_{d}} ) ) \qquad ( 3 )$$

$${L}_{k} = \{ \begin{matrix}{N}_{( {w}_{dn} ) k \backslash {z}_{dn}} + β & if k ∈ \{ 1 … K \} \\ {ϕ}_{{kw}_{dn}} & if k ∈ \{ K + 1 … K + V \}\end{matrix}$$

In equation (3), the upper term of Lₖ represents a term corresponding to conventional topic estimation, and the lower term of Lₖ represents a term corresponding to the NC samples.

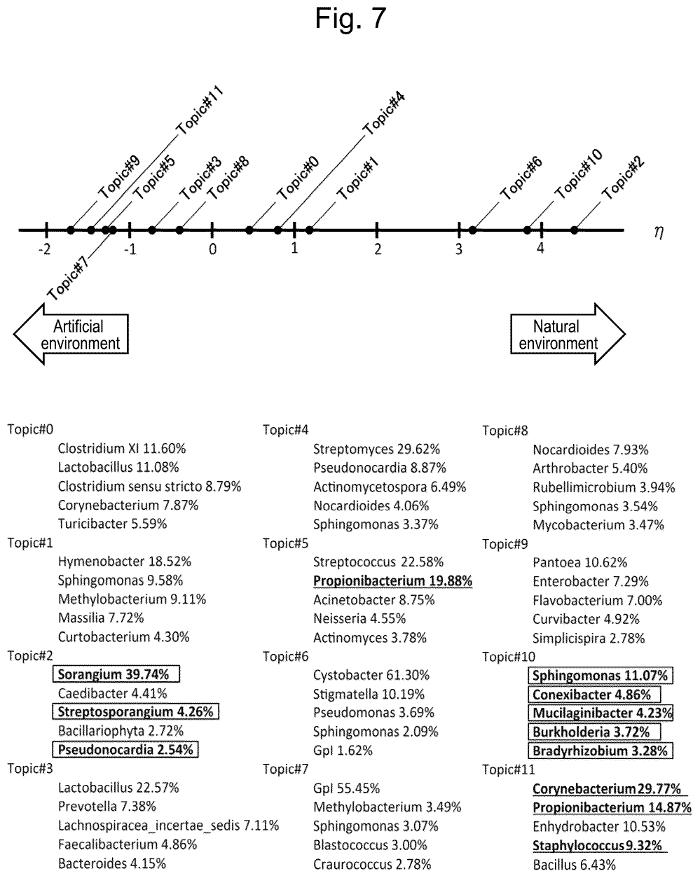

FIG. 7 illustrates topics and η parameters extracted by machine learning related to the BPS estimation model.

In the BPS estimation model, the microbial community structure is partitioned into a set of subcommunities. One of sub-communities is derived from humans, while another is derived from the natural environment. These subcommunities are the topics estimated in the model. In a sample collected from the air, these topics are present in a mixed manner. The way topics are mixed (which topics are dominant and how dominant they are) is varied depending on the sample. Further, not all of the microbes as the members of the topics are observed in the sample; instead, the results of sampling performed stochastically in accordance with the community structures (types of microbes and abundance thereof) of the topics are observed.

Also, each sample has a BPS calculated independently of the microbial community structure data. In the BPS estimation model, it is assumed that the BPS is defined according to “how the topics are mixed (mixing proportions)” for each sample. For example, a certain topic has a negative influence on the BPS (influence to decrease the BPS), while another certain topic has a positive influence on the BPS (influence to increase the BPS). The parameter representing the influence of each topic on the increase or decrease of the BPS is the n parameter. In the BPS estimation model, it is assumed that the BPS of each sample is calculated according to the inner product of the mixing proportions of topics (topic composition) in each sample and the η parameter.

In the present embodiment, 585 samples collected from the air in the reference spaces are prepared, and the microbial community structure data and the BPS of each sample were acquired. Further, as the NC samples, 27 samples were prepared, and their microbial community structure data was acquired. These data were set in the estimation model and machine learning was performed, whereby 12 topics of Topic #0 to Topic #11 were extracted. The number of topics extracted (12) was set after verifying in advance that a further increase in the number of topics extracted would not significantly increase the model's estimation accuracy.

FIG. 7 shows a number line on which the η parameters of the extracted Topic #0 to Topic #11 are plotted, and the top five microbes belonging to each of the topics and their abundance. With reference to FIG. 7, it is seen that the topics of which the η parameter is negative, such as Topic #5 and Topic #11, tend to include large amounts of human-derived microbes, marked with underlines, such as “Propionibacterium”, which is a human commensal bacterium. With further reference to FIG. 7, it is seen that the topics of which the n parameter is positive, such as Topic #2 and Topic #10, tend to include large amounts of naturally derived microbes, marked in boxes, such as “Sorangium”, which is a soil bacterium. That is, it may be considered that the topics having negative η parameters have a large negative influence on the BPS, while the topics having a positive η parameter have a large positive influence on the BPS. Accordingly, it may be considered that the greater the mixing proportions of topics with negative η parameters, the closer the space of the microbial community structure data is to an artificial environment, and the greater the mixing proportions of topics with positive η parameters, the closer the space of the microbial community structure data is to a natural environment.

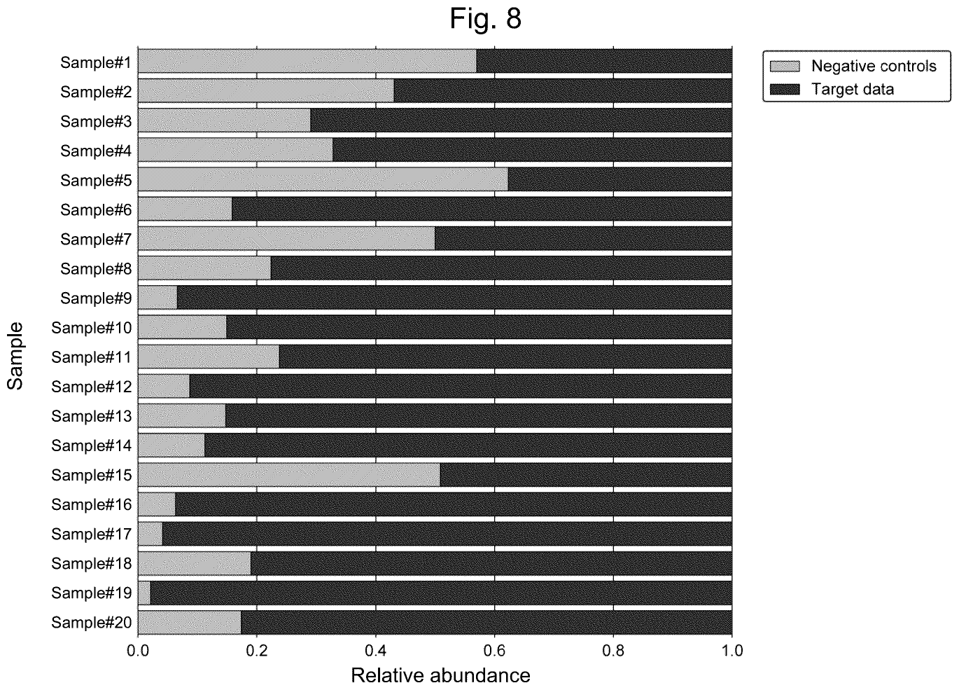

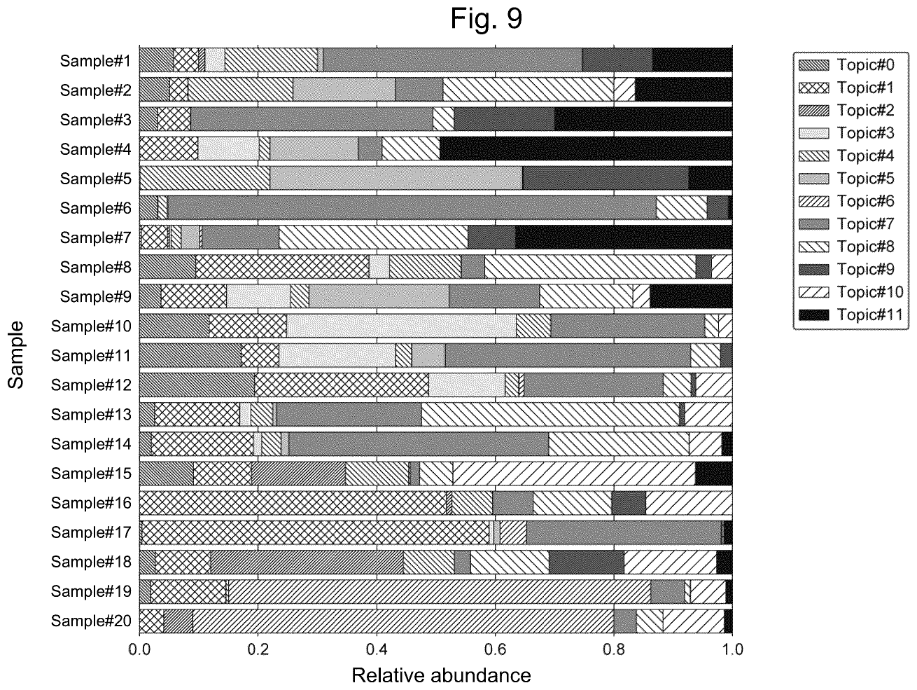

FIG. 8 illustrates the contaminated proportions of the microbial community structure data of the NC samples that contaminates the microbial community structure data of each sample. FIG. 9 illustrates the mixing proportions of topics in each sample illustrated in FIG. 8.

The graph of FIG. 8 indicates the estimated contaminated proportions for 20 samples randomly selected from the (585) samples used for training models. In FIG. 8, “Target data” indicates the proportion (relative abundance) of the microbial community structure data of each sample, and “Negative controls” indicates the proportions (relative abundance) of the microbial community structure data of the NC samples. The graph shown in FIG. 9 indicates the result of removing the “Negative controls” from FIG. 8 and then calculating the mixing proportions of the topics (relative abundance) in each sample relative to 100% of the “Target data” portion. Further, in the graphs shown in FIG. 8 and FIG. 9, the samples are arranged in order of increasing BPS from the top of the drawing figures.

As in the samples of “Sample #1” and “Sample #5” illustrated in FIG. 8, there are samples of which the contaminated proportion of the microbial community structure data of the NC samples is more than 50%. Accordingly, in order to accurately extract the mixing proportion of each topic in each sample, it is preferable to have the microbial community structure data of the NC samples removed from the microbial community structure data of each sample.

As illustrated in FIG. 9, it is seen that the samples with smaller BPS tend to include more topics having a negative η parameter, such as Topic #5 and Topic #11. It is also seen that the samples with larger BPS tend to include more topics having a positive η parameter, such as Topic #2 and Topic #10. According to FIG. 7 to FIG. 9, it can be said that the BPS estimation model of the present embodiment is capable of extracting topics in accordance with the BPS.

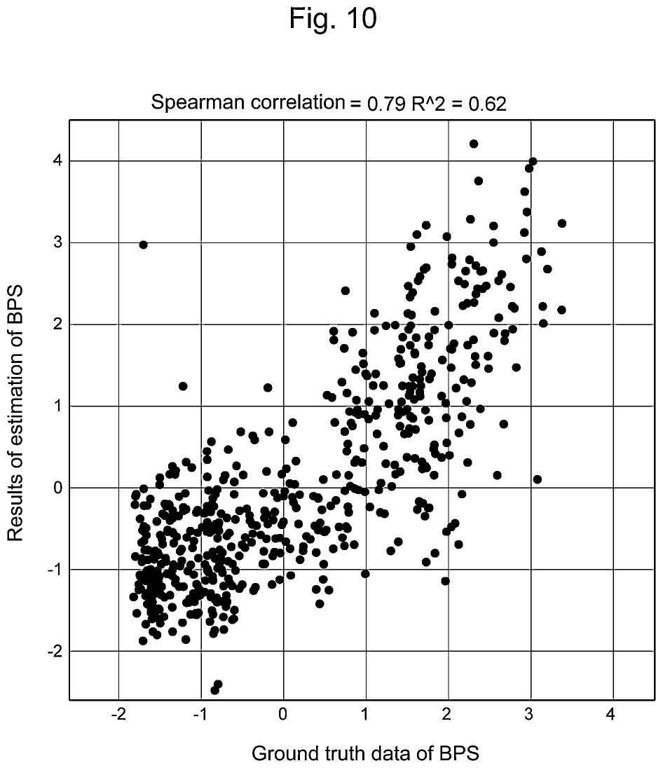

FIG. 10 illustrates the result of the validation of the BPS estimation model.

In the present embodiment, the prediction accuracy of the model was estimated by 5-fold cross validation. Specifically, first, the data sets (microbial community structure data and BPS) of the 585 samples were divided into five data set. Of the divided five data set, four were used for model training, and the remaining one was used for testing to estimate the accuracy of the model by predicting BPS estimates and comparing them to the ground truth BPS. This process was repeated five times to verify the estimation model.

When estimating the BPS by inputting the test data into the trained estimation model, first, the parameters of the estimation model were used to estimate the mixing proportions of the topics (topic composition) in each test data from the microbial community structure data of the test data. Thereafter, the product of the mixing proportions of the topics in each test data and the n parameter is calculated and converted into a BPS.

The graph shown in FIG. 10 indicates the result of computation of the Spearman correlation between the BPS estimation result by the test data and the ground truth data. The vertical axis of FIG. 10 indicates the BPS estimation result by the test data, and the horizontal axis of FIG. 10 indicates the ground truth data. The dots in FIG. 10 indicate the test samples. The Spearman correlation between the BPS estimation result by the test data and the ground truth data indicates a high value of about 0.79. This result clearly demonstrated that the BPS estimation model of the present embodiment possesses a high level of predictive accuracy.

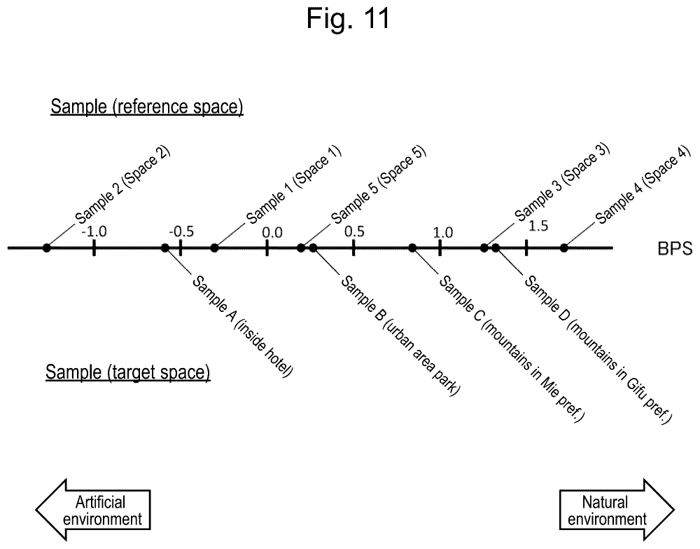

FIG. 11 illustrates the result of estimating the BPS of target spaces using the BPS estimation model.

FIG. 11 illustrates a number line of the BPS. On the upper side of the number line shown in FIG. 11, the samples collected in the respective reference spaces are plotted, as in FIG. 3. On the lower side of the number line shown in FIG. 11, the samples collected in the respective target spaces are plotted. The samples collected in the target spaces are unknown samples not used for the training of the BPS estimation model. The microbial community structure data of each of the samples collected in the target spaces was input into the trained BPS estimation model, and the BPS of the target spaces were estimated. For the sample A collected inside a hotel, a negative (left side) BPS was estimated, indicating a space closer to an artificial environment. For the sample B collected in an urban-area park, a BPS indicating a space in between an artificial environment and a natural environment was estimated. For the sample C collected in the mountains of Mie Prefecture, a positive (right side) BPS indicating a space closer to a natural environment was estimated. For the sample D collected in the mountains of Gifu Prefecture, a positive (right side) BPS indicating a space even closer to a natural environment than the sample C was estimated.

### Effects

As described above, the space evaluation system 1 of the present embodiment includes the setting unit 12 in which the naturalness (BPS) as an index of how close a space is to a natural environment is set. The space evaluation system 1 of the present embodiment further includes the estimating unit 11 which estimates, from the air quality data indicating the type of materials including microbes included in a sample collected from the air in a target space to be evaluated and indicating the abundance of each of the materials (microbial community structure data), the naturalness (BPS) of the target space from which the sample has been collected.

Thus, the space evaluation system 1 of the present embodiment, by simply collecting a sample from the air in a target space that may be freely determined, and acquiring the air quality data of the collected sample, can estimate naturalness from the air quality data alone. That is, the space evaluation system 1 of the present embodiment can estimate naturalness from the air quality data alone, without capturing an image of the target space from above, acquiring physiological response information in the target space, or performing sensory evaluation each time. In addition, the space evaluation system 1 of the present embodiment is applicable whether the target space is a space having no soil, such as an indoor space, or an outdoor space closer to a natural environment, and can estimate naturalness from the air quality data alone irrespective of the attributes of the target space. Conventionally, there have been examples in which the contamination degree of air is expressed as an index and evaluated in terms of inorganic gases, volatile organic compounds and the like. However, air quality data has not been used for evaluating naturalness. Naturally, there is no previous example of a model for estimating naturalness from air quality data. The space evaluation system 1 of the present embodiment can estimate naturalness only from the air quality data of the target space that may be freely determined. Accordingly, the space evaluation system 1 of the present embodiment can simply and quantitatively evaluate how close an unknown space is to a natural environment.

Further, in the space evaluation system 1 of the present embodiment, the machine learning related to the naturalness estimation model constituting the estimating unit 11 is performed by sLDA, which is one of topic models.

Thus, the space evaluation system 1 of the present embodiment can extract, for example, the structure (i.e., topics) of a sub-community that exists in microbial community structure data and affects naturalness. Accordingly, the space evaluation system 1 of the present embodiment can more accurately estimate naturalness by means of the estimating unit 11, and can therefore more accurately evaluate how close an unknown space is to a natural environment.

As noted above, as a machine learning approach related to estimation model, machine learning approaches such as random forest, deep learning and the like are applicable. However, for example, with such approaches, it is not easy to extract the structure of a sub-community that exists in microbial community structure data and that affects naturalness. Further, for example, because the process of acquiring microbial community structure data is essentially a process of sampling from a “true microbial community”, inclusion of stochastic fluctuation of data as noise cannot be avoided. With deterministic approaches such as deep learning, it is not easy to capture probabilistic property of data, and it is not easy to perform modelling of a probabilistic sampling process explicitly. In addition, for example, depending on the microbial community structure data, sampling may not be fully accomplished and there may be much sparse data. Accordingly, with a deterministic approach such as deep learning, it may be also difficult to select a regularization means for preventing over-training. For these reasons, for the estimation model, it is effective to use the approach using sLDA of the present embodiment which is a stochastic model, is capable of extracting the structure of a sub-community, and is a modeling approach that learns regression to numerical value information.

In addition, because the space evaluation system 1 of the present embodiment is capable of extracting topics affecting naturalness as described above, it can be clearly shown what topics should be added or removed to change naturalness. Thus, with the space evaluation system 1 of the present embodiment, it is possible to simply and quantitatively ascertain the types and abundance of materials related to air quality necessary for obtaining desired naturalness. Accordingly, with the space evaluation system 1 of the present embodiment, it is possible to simply and quantitatively develop a guideline for designing a space having desired naturalness.

### Another Embodiment Concerning Negative Control

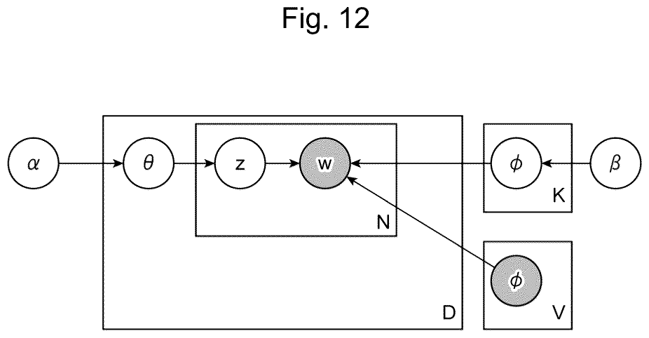

With reference to FIG. 12 to FIG. 14, another embodiment concerning negative control will be described.

In the foregoing embodiment, the BPS estimation model constituting the estimating unit 11 uses the above-described data set (microbial community structure data and BPS) and the microbial community structure data of an NC sample to perform machine learning by sLDA. The trained estimation model estimates the contaminated proportion of the microbial community structure data of the NC sample that contaminates the microbial community structure data of the sample collected in the target space, and estimates the BPS of the target space from the microbial community structure data of the target space from which the microbial community structure data of the NC sample has been excluded.

Here, the model itself for estimating the contaminated proportion of the microbial community structure data of the NC sample (hereafter also referred to as “NC estimation model”) can be constructed according to an approach different from the sLDA illustrated in FIG. 6. In the present embodiment, as the machine learning approach related to the NC estimation model, an approach is adopted in which conventional (unsupervised) latent Dirichlet allocation (hereafter also referred to as “LDA”), which is one of topic models, is extended. Specifically, as a machine learning approach related to the NC estimation model, an approach (hereafter also referred to as “LDAnc”) is adopted in which computational expressions for estimating the contaminated proportion of the microbial community structure data of the NC sample is added to conventional LDA.

FIG. 12 illustrates a graphical model representing the NC estimation model by LDAnc.

The variables used in the mathematical expressions for describing the NC estimation model by LDAnc are similar to those described above with reference to FIG. 6. The microbial community structure data of an NC sample is acquired in advance by, as described above with reference to FIG. 5, performing a metagenome analysis with respect to microbes present in the collecting device, the analysis device, the reagents and the like, and clarifying the phylogenetic composition of the microbes. In LDAnc, while the community structure of the NC sample is fixed, it is assumed that the community structures of topics are unknown, and parameters are updated by Gibbs sampling, as in conventional LDA. LDAnc is an approach that combines the advantage of LDA for estimating an unknown sub-community, and the advantage of Source Tracker for estimating the mixing proportions of a known sub-community.

The generative process the NC estimation model is as follows:
1. θ_{d}˜Dir(α): Topic composition θ_{d} of sample d is sampled from Dirichlet distribution Dir(α) having α as a parameter.
2. ϕₖ˜Dir(β): Community structure ϕₖ of topic k is sampled from Dirichlet distribution Dir(β) having β as a parameter.
3. Concerning DNA sequence n of sample d
3-1. z_{dn}˜Categorical(θ_{d}): Topic number or NC sample number of a DNA sequence is sampled from a categorical distribution.
3-2. w_{dn}˜Categorical(ϕₖ): Microbial strain is sampled from corresponding topic or NC sample.

Bayesian inference of an unknown parameter is performed. As in conventional LDA, all unknown parameters are inferred by collapsed Gibbs sampling. As initial values, number k∈{1, . . . , K, K+1, . . . , K+V} corresponding to any of K topics or V NC samples is randomly assigned. Gibbs sampling is repeated until the joint probability of the entire model converges. In this case, the community structure ϕ_{k=K+1 . . . K+V} of the NC samples is fixed and is not updated during the repetition, which is in contrast to conventional LDA.

The full conditional distribution of the topic z_{dn} of DNA sequence n of the sample d is described as follows:

$$p ( {z}_{dn} = k \vert {z}_{\backslash dn} , w ) \qquad ( 4 )$$

$$∝ {L}_{k} \times \frac{{N}_{dk \backslash {z}_{dn}} + α}{{N}_{d \backslash {z}_{dn}} + K α}$$

$${L}_{k} = \{ \begin{matrix}\frac{{N}_{( {w}_{dn} ) k \backslash {z}_{dn}} + β}{{N}_{k \backslash {z}_{dn}} + T β} & if k ∈ \{ 1 … K \} \\ {ϕ}_{{kw}_{dn}} & if k ∈ \{ K + 1 … K + V \}\end{matrix}$$

Finally, the number assigned to each DNA sequence is examined, and the DNA sequences to which the numbers corresponding to the NC samples are assigned are identified. Then, of the entire DNA sequences in the sample, the proportions occupied by the DNA sequences to which the numbers corresponding to the NC samples are assigned are computed. In this way, the contaminated proportion of the NC samples can be estimated.

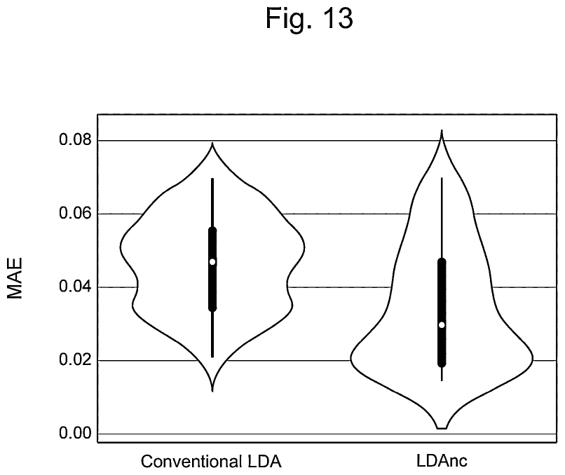

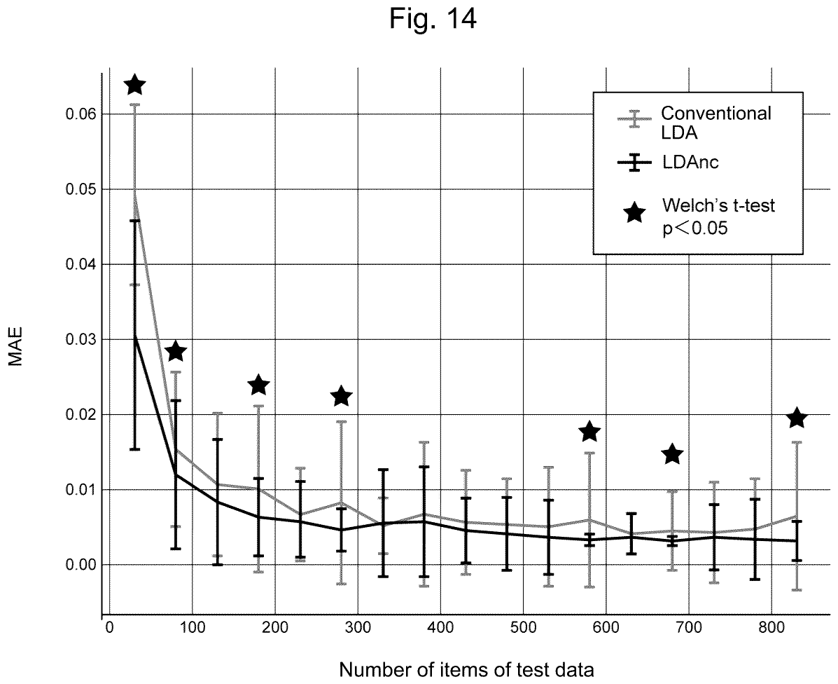

FIG. 13 illustrates an example of the result of verifying the estimation accuracy of the NC estimation model by LDAnc. FIG. 14 illustrates another example of the result of verifying the estimation accuracy of the NC estimation model by LDAnc.

The model validation was performed in a simulated manner using images. Specifically, 10 images were prepared as ground truth data, and 30 images were prepared as test data. The 10 images of the ground truth data comprised patterns of predetermined colors and shapes corresponding to sub-communities disposed in different pixel regions in each image. The 30 images of the test data comprised the patterns corresponding to the sub-communities randomly mixed in the images. Then, the NC estimation model by LDAnc and an NC estimation model by conventional LDA were used to estimate the patterns of the ground truth data from the test data. In this case, in the NC estimation model by conventional LDA, the patterns of the ground truth data were estimated assuming that the 10 items of the ground truth data were all unknown. In the NC estimation model by LDAnc, the patterns of the ground truth data were estimated assuming that of the 10 items of the ground truth data, two were known and the remaining eight were unknown. Then, a mean absolute error (hereafter also referred to as “MAE”) between the estimated patterns and the patterns of the ground truth data was calculated. Such process was repeated 100 times to determine the distribution of the MAE in each NC estimation model.

FIG. 13 shows the distribution of the MAE in the respective NC estimation models. It is seen that the MAE of the NC estimation model by LDAnc is smaller than that of the NC estimation model by conventional LDA. Thus, it is seen that the NC estimation model by LDAnc provides higher estimation accuracy than the NC estimation model by conventional LDA.

FIG. 14 shows the MAE values in each NC estimation model when the number of test data items was changed. It is seen that the MAE of the NC estimation model by LDAnc is generally smaller than that of the NC estimation model by conventional LDA. Thus, it is seen that the NC estimation model by LDAnc provides higher estimation accuracy than the NC estimation model by conventional LDA. In particular, it is seen that when the number of test data items is small, the MAE of the NC estimation model by LDAnc is notably smaller than that of the NC estimation model by conventional LDA. Thus, it is seen that the NC estimation model by LDAnc is more effective than the NC estimation model by conventional LDA particularly when the number of test data items is small. Further, it is seen that the MAE of the NC estimation model by LDAnc has less variation in accordance with a change in the number of test data items than the NC estimation model by conventional LDA. Thus, it is seen that the NC estimation model by LDAnc provides more stable estimation accuracy than the NC estimation model by conventional LDA.

Thus, with the NC estimation model by LDAnc, it is possible to estimate the contaminated proportion of the microbial community structure data of NC samples that contaminates the microbial community structure data of the sample collected in the target space with higher estimation accuracy than by the NC estimation model by conventional LDA. With the NC estimation model by LDAnc, it is possible to acquire the true microbial community structure data of the collected sample by subtracting the estimated contaminated proportion of the microbial community structure data of the NC samples from the microbial community structure data of the sample collected in the target space.

It is noted that the NC estimation model by LDAnc is not limited to microbial community structure data and may be applied to count data other than microbial community structure data, such as air quality data and document data. The NC estimation model by LDAnc may constitute a part of the estimating unit 11 provided in the arithmetic processing device 10 of the space evaluation system 1.

While embodiments of the present invention have been described, the present invention is not limited to the foregoing embodiments, and various design changes may be made without departing from the spirit and scope of the claims. In the present invention, the configuration of a certain embodiment may be added to the configuration of another embodiment, the configuration of the certain embodiment may be substituted with another embodiment, or a part of the configuration of the certain embodiment may be deleted.

### REFERENCE SIGNS LIST

1 Space evaluation system
10 Arithmetic processing device
11 Estimating unit
12 Setting unit

1. A space evaluation system comprising:
a setting unit in which naturalness as an index of how close a space is to a natural environment is set; and
an estimating unit to estimate, from air quality data indicating a type of material including a microbe included in a sample collected from air in a target space to be evaluated and indicating an abundance of each material, the naturalness of the target space from which the sample has been collected.

2. The space evaluation system according to claim 1, wherein in the setting unit, the naturalness is set based on environment data indicating conditions of a plurality of specific spaces,
the environment data including data acquired in each of the plurality of specific spaces having different environments.

3. The space evaluation system according to claim 2, wherein the environment data includes quantitative data acquired by a sensor in the specific spaces and qualitative data acquired in the specific spaces through sensory evaluation.

4. The space evaluation system according to claim 2, wherein the estimating unit has machine-learned to calculate the naturalness with respect to the air quality data of the target space, using, as training data, a data set in which the air quality data of a sample for learning collected from air in each of the plurality of specific spaces is associated with the naturalness corresponding to each of the plurality of specific spaces.

5. The space evaluation system according to claim 1, wherein:
the air quality data is acquired by analyzing, by means of an analysis device, a sample collected by a collecting device;
in the setting unit, one or both of the air quality data of the material present in the collecting device before the sample is collected and the air quality data of the material present in the analysis device before the sample is analyzed are set as the air quality data of a negative control sample; and
the estimating unit estimates a contaminated proportion of the air quality data of the negative control sample that contaminates the air quality data of the sample collected in the target space, and estimates the naturalness of the target space from the air quality data of the target space from which the air quality data of the negative control sample has been removed.

6. The space evaluation system according to claim 3, wherein the estimating unit has machine-learned to calculate the naturalness with respect to the air quality data of the target space, using, as training data, a data set in which the air quality data of a sample for learning collected from air in each of the plurality of specific spaces is associated with the naturalness corresponding to each of the plurality of specific spaces.

7. The space evaluation system according to claim 2, wherein:
the air quality data is acquired by analyzing, by means of an analysis device, a sample collected by a collecting device;
in the setting unit, one or both of the air quality data of the material present in the collecting device before the sample is collected and the air quality data of the material present in the analysis device before the sample is analyzed are set as the air quality data of a negative control sample; and
the estimating unit estimates a contaminated proportion of the air quality data of the negative control sample that contaminates the air quality data of the sample collected in the target space, and estimates the naturalness of the target space from the air quality data of the target space from which the air quality data of the negative control sample has been removed.

8. The space evaluation system according to claim 3, wherein:
the air quality data is acquired by analyzing, by means of an analysis device, a sample collected by a collecting device;
in the setting unit, one or both of the air quality data of the material present in the collecting device before the sample is collected and the air quality data of the material present in the analysis device before the sample is analyzed are set as the air quality data of a negative control sample; and
the estimating unit estimates a contaminated proportion of the air quality data of the negative control sample that contaminates the air quality data of the sample collected in the target space, and estimates the naturalness of the target space from the air quality data of the target space from which the air quality data of the negative control sample has been removed.

9. The space evaluation system according to claim 4, wherein:
the air quality data is acquired by analyzing, by means of an analysis device, a sample collected by a collecting device;
in the setting unit, one or both of the air quality data of the material present in the collecting device before the sample is collected and the air quality data of the material present in the analysis device before the sample is analyzed are set as the air quality data of a negative control sample; and
the estimating unit estimates a contaminated proportion of the air quality data of the negative control sample that contaminates the air quality data of the sample collected in the target space, and estimates the naturalness of the target space from the air quality data of the target space from which the air quality data of the negative control sample has been removed.
